(19) United States
(12) Patent Application Publication
(10) Pub. No.: US 2024/0044716 A1
(43) Pub. Date: Feb. 8, 2024

(54) SENSOR AND METHOD FOR COMMISSIONING SUCH A SENSOR

(71) Applicant: Aktlebolaget SKF, Goteborg (SE)

(72) Inventors: Simon INNES, Aberdeen (GB); Carl-Axel Kreku, Lulea (SE); Christopher Gwyn James, Houten (NL)

(21) Appl. No.: 18/358,116

(22) Filed: Jul. 25, 2023

(30) Foreign Application Priority Data
Aug. 3, 2022 (DE) 102022208040.8

(51) Int. Cl. G01K 1/14

(52) U.S. Cl. CPC G01K 1/14

(57) ABSTRACT

A Sensor (2) is proposed. The sensor (2) includes a memory (5) and a control means (6). The control means (6) is configured to: store in the memory (5) connection parameters (5a), switch the sensor (2) in a sleep mode when connection parameters (5a) are stored in the memory (5) and when receiving a sleep mode signal, and start the sensor at the reception of a start signal (S1) by the control means of the sensor (2).

### CROSS-REFERENCE TO RELATED APPLICATION

This application claims priority to German Application No. 102022208040.8, filed Aug. 3, 2022, the entirety of which is hereby incorporated by reference.

### FIELD

The present disclosure is directed to a sensor and a method for commissioning such a sensor.

More particularly, the disclosure deals with commissioning a sensor offsite such that the sensor is not commissioned after being installed in an area with restrictions such an atmospheres explosives (ATEX) area.

### BACKGROUND

Some sensors are certified ATEX so that they may be installed in an ATEX environment.

After installation of the sensors in the ATEX environment, sensors are commissioned in the ATEX environment.

However, to commission such sensors, a device is needed which is generally not certified for an ATEX environment.

The device may be a mobile phone comprising a near field communication (NFC) interface communicating with an NFC interface of sensors.

Such a mobile phone is not allowed to enter an ATEX area so that the sensors cannot be commissioned.

Further, in some industries, the person responsible for commissioning sensors and the person responsible for the installation of the sensors in the ATEX environment are different.

Generally, the tasks that should be done by each person are not clearly define.

Further, commissioning sensors in an industrial environment may be complex and may be dangerous according to for example noises in the industrial environment generated for example by machines, the temperature values in the industrial environment.

Consequently, the present disclosure intends to overcome these disadvantages.

### SUMMARY

According to an aspect, a method commissioning a sensor is proposed.

The method comprises:
storing connection parameters in a memory of the sensor,
sending a sleep mode signal to control means of the sensor to switch the sensor in a sleep mode when the connection parameters are stored in the memory of the sensor,
installing the sensor in sleep mode in an area in which the sensor measures a value,
sending a start signal to the control means of the sensor when the sensor in sleep mode is installed in the area, and
starting the sensor at the reception of the start signal by the control means of the sensor.

The sensor may be commissioned offsite in a zone not subject to restrictions and is switch in a sleep mode after commissioning.

The sensor is easily activated after installation in the area subjected for example to restrictions.

Preferably, sending a start signal to the control means of the sensor comprises tagging the sensor in sleep mode with a device.

Advantageously, starting the sensor comprises:
checking by the control means if the connection parameters are saved in the memory of the sensor, and
if the connection parameters are saved in the memory, establishing a connection between the control means of the sensor and a gateway to transfer measurement measured by measurement means of the sensor.

Preferably, the area is an ATEX area, the sensor being ATEX certified.

According to another aspect, a sensor is proposed.

The sensor comprises:
a memory,
control means configured to:
store in the memory connection parameters,
switch the sensor in a sleep mode when connection parameters are stored in the memory and when receiving a sleep mode signal, and
starting the sensor at the reception of a start signal by the control means of the sensor.

Advantageously, the sensor comprises communication means configured to receive and deliver to the control means the connection parameters, the sleep mode signal, and the start signal.

Preferably, the communication means are configured to communicate with a NFC device delivering the start signal.

Advantageously, the control means are configured during starting of the sensor to:
check if the connection parameters are stored in the memory of the sensor, and
if the connection parameters are saved in the memory, establishing a connection between the control means of the sensor and a gateway to transfer measurement measured by measurement means of the sensor

Preferably, the sensor is ATEX certified.

According to another aspect, an area comprising a sensor as defined above is proposed.

### BRIEF DESCRIPTION OF THE DRAWINGS

Other advantages and features of the disclosure will appear on examination of the detailed description of embodiments, in no way restrictive, and the appended drawings in which:

FIG. 1 illustrates schematically a sensor in an area according to the disclosure; and

FIG. 2 illustrates schematically a method for commissioning the sensor according to the disclosure.

### DETAILED DESCRIPTION

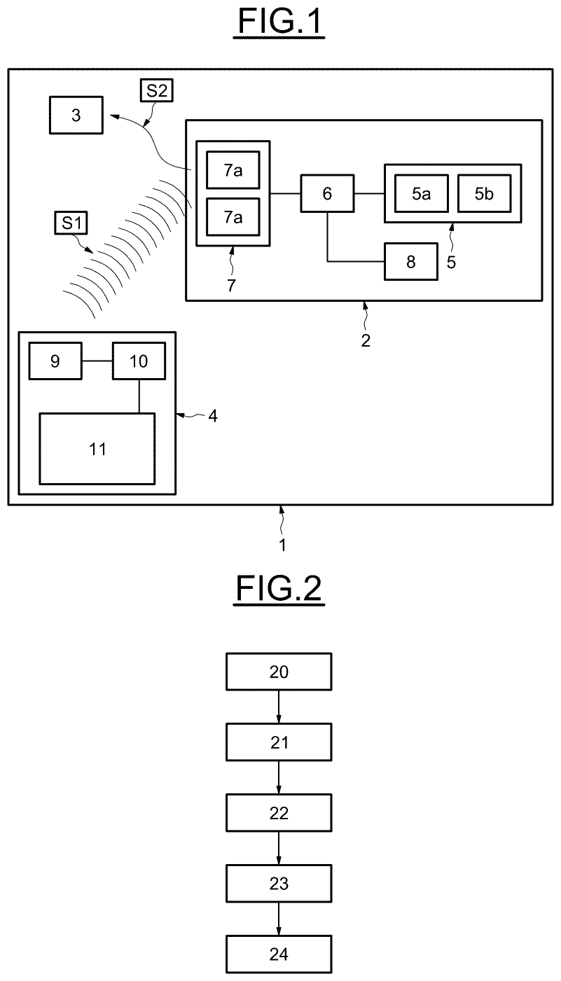

Reference is made to FIG. 1 which represents an area comprising for example an ATEX industrial installation 1.

A sensor 2 is implemented in the installation 1 for example to measure the temperature inside the installation 1.

The sensor 2 is ATEX certified.

In variant, the sensor 2 may not be ATEX certified and is implemented in a non ATEX environment.

The sensor 2 may measure others parameters of the installation 1, for example humidity.

The installation 1 further comprises a gateway 3 communicating with the sensor 2 to transfer the measured values to a network (not represented).

In variant, the gateway 3 is outside the installation 1.

A device 4 is introduce in the installation 1 to start the sensor 2 as exposed in the following and is ATEX certified.

In variant, if the sensor 2 is ATEX certified and is implemented in a non ATEX environment, the device 4 may be not ATEX certified.

The sensor 2 comprises a memory 5, control means 6, communication means 7, and measurement means 8.

The control means 6 may comprise a processing unit to implement the memory 5, the communication means 7 and the measurement means 8.

The communication means 7 comprise for example first communication means 7a communicating wirelessly with the gateway 3, and second communication means 7b comprising for example a near field communication NFC interface 7b.

In variant, first communication means 7a and the gateway 3 are connected together with a wired connection, for example an optical fiber.

The measurement means 8 comprise for example a measurement interface measuring the temperature inside the installation 1.

The memory 5 saves connection parameters 5a and at least one algorithm 5b.

The algorithm 5b is implemented by the control means 6 to condition the values measured by the measurement means 8 in a standard allowing their transmission by the communication means 7a.

The connection parameters 5a comprise the identification of the gateway 3 so that the first communication means 7a send the conditioned values to the gateway 3.

The device 4 comprises communication means 9 communicating with the second communication means 7b of the sensor 2, command means 10, and a human machine interface 11 comprising for example a touchscreen.

The communication means 9 comprise for example an NFC interface.

The command means 10 comprise for example a processing unit implementing the communication means 9 and the human machine interface 11.

The device 4 may be an autonomous portable device.

FIG. 2 illustrates an example of a method for commissioning the sensor 2.

It is assumed that the algorithm 5b is stored in the memory 5. and the sensor 2 is not installed in the ATEX industrial installation 1 so that the sensor 2 is offsite, for example in an office.

In step 20, the connection parameters 5a are stored in the memory 5.

The connection parameters 5a are for example transmitted to the sensor 2 through the communication means 7 by the mobile phone.

In step 21, when the connection parameters are stored in the memory 5 of the sensor 2, at the reception of a sleep mode signal sent by the mobile phone, the control means 6 switch the sensor 2 in a sleep mode.

In step 22, the sensor 2 in sleep mode is installed in the ATEX industrial installation 1.

In step 23, when the sensor in sleep mode is installed in the ATEX industrial installation 1, the device 4 is introduced into the ATEX industrial installation 1.

An operator commands the device 4 with the human machine interface 11 so that the communication means 9 of the device 4 delivers a start signal S1.

As the communication means 9 of the device 4 and the sensor 2 and the second communication means 7b of the sensor 2 comprise NFC interfaces, the device 4 and the sensor 2 are tagged.

At step 24, at the reception of the start signal S1 by the control means 6 through the second communication means 7b, the control means 6 start the sensor 2.

The control means 6 check if the connection parameters 5a are stored in the memory 5 of the sensor 2.

If the connection parameters 5a are saved in the memory 5, the control means 6 establish a connection with the gateway 3 to transfer values measured by the measurement means 8 of the sensor 2.

The measurement means 8 measure temperature values and the first communication means 7b send a second signal S2 comprising the measured temperature values to the gateway 3.

The sensor 2 may be commissioned offsite in a zone not subject to restrictions and the connection parameters are saved in the sensor 2 and is switch in a sleep mode after commissioning reducing for example the power consumption of the sensor 2 comprising a battery to supply the control means 6.

After installation of the sensor 2, the sensor 2 is waked up after receiving only the start signal S1 delivered by the device 4.

Only the device 4 needs to be ATEX certified.

Further, as the steps 20,21 of configuring the connection parameters of the sensor and steps of installing the sensor 2 (steps 23, 24) are clearly split, the tasks that should be done by a first person for configuring the sensor 2 and the tasks that should be done by a second person for installing the sensor 2 in the ATEX industrial installation 1 are clearly define in order to ease the installation steps.

What is claimed is:

1. A method for commissioning a sensor, the method comprising:
storing connection parameters in a memory of the sensor,
sending a sleep mode signal to control means of the sensor to switch the sensor in a sleep mode when the connection parameters are stored in the memory of the sensor,
installing the sensor in sleep mode in an area in which the sensor measures a value,
sending a start signal to the control means of the sensor when the sensor in sleep mode is installed in the area, and
starting the sensor at the reception of the start signal by the control means of the sensor.

2. The method according to claim 1, wherein sending a start signal to the control means of the sensor comprises tagging the sensor in sleep mode with a device.

3. The method according to claim 1, wherein starting the sensor comprises:
checking by the control means if the connection parameters are stored in the memory of the sensor, and
if the connection parameters are stored in the memory, establishing a connection between the control means of the sensor and a gateway to transfer measurement measured by measurement means of the sensor.

4. The method according to claim 3, wherein the area is an ATEX area, the sensor being ATEX certified.

5. The method according to claim 2, wherein starting the sensor comprises:
checking by the control means if the connection parameters are stored in the memory of the sensor, and
if the connection parameters are stored in the memory, establishing a connection between the control means of the sensor and a gateway to transfer measurement measured by measurement means of the sensor.

6. The method according to claim 5, wherein the area is an ATEX area, the sensor being ATEX certified.

7. A sensor comprising:
a memory,
control means configured to:
store in the memory connection parameters,
switch the sensor in a sleep mode when connection parameters are stored in the memory and when receiving a sleep mode signal, and
starting the sensor at the reception of a start signal by the control means of the sensor.

8. The sensor according to claim 7, further comprising communication means configured to receive and deliver to the control means the connection parameters, the sleep mode signal, and the start signal.

9. The sensor according to claim 8, wherein the communication means are configured to communicate with a NFC device delivering the start signal.

10. The sensor according to claim 7, wherein the control means are configured during starting of the sensor to:
check if the connection parameters are stored in the memory of the sensor, and
if the connection parameters are saved in the memory, establishing a connection between the control means of the sensor and a gateway to transfer measurement measured by measurement means of the sensor.

11. The sensor according to claim 7, wherein the sensor is ATEX certified.

12. An area comprising a sensor according to claim 7.

13. The sensor according to claim 8, wherein the control means are configured during starting of the sensor to:
check if the connection parameters are stored in the memory of the sensor, and
if the connection parameters are saved in the memory, establishing a connection between the control means of the sensor and a gateway to transfer measurement measured by measurement means of the sensor.

14. The sensor according to claim 13, wherein the sensor is ATEX certified.

15. An area comprising a sensor according to claim 13.

16. The sensor according to claim 9, wherein the control means are configured during starting of the sensor to:
check if the connection parameters are stored in the memory of the sensor, and
if the connection parameters are saved in the memory, establishing a connection between the control means of the sensor and a gateway to transfer measurement measured by measurement means of the sensor.

17. The sensor according to claim 16, wherein the sensor is ATEX certified.

18. An area comprising a sensor according to claim 16.
